(19) United States
(12) Patent Application Publication
(10) Pub. No.: US 2009/0312637 A1
(43) Pub. Date: Dec. 17, 2009

(54) ULTRASOUND MONITORING AND FEEDBACK FOR MAGNETIC HYPERTHERMIA

(75) Inventors: Balasundara Raju, Tarrytown, NY (US); David Savery, Calas (FR)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/997,510

(22) PCT Filed: Jul. 12, 2006
(86) PCT No.: PCT/IB06/52371

§ 371 Date: Jan. 31, 2008

Related U.S. Application Data
(60) Provisional application No. 60/705,215, filed on Aug. 3, 2005.

(51) Int. Cl. A61B 8/00; A61M 5/00; A61B 18/18

(52) U.S. Cl. 600/439; 600/458; 604/22; 607/103; 977/929

(57) ABSTRACT

A method and system of magnetic hyperthermia control using ultrasound thermometry, the method comprising: acquiring a reference set of ultrasound data corresponding to a tissue of interest (121); applying a plurality of magnetic nanoparticles (210) at the tissue of interest (121); applying an electromagnetic field based on a set of operating parameters to initiate the hyperthermia; acquiring another set of ultrasound data corresponding to the tissue of interest (121); and determining a temperature change based on the reference set of ultrasound data and the other set of ultrasound data.

The present disclosure is directed to a methodology and system for monitoring and controlling magnetic hyperthermia using ultrasound measurement techniques.

Hyperthermia is an old therapeutic method which has been carried out from ancient Greece. Hyperthermia is a method in which malignant tumor, tissue, or otherwise is entirely heated to kill the malignant tumor cells preferably without damaging the adjacent noncancerous cells.

Magnetic hyperthermia is a method in which magnetic fine particles (nanoparticles) such as magnetite (Fe₃O₄) are used as an internal heating element, and the magnetic fine particles are heated by a time-varying magnetic field. Furthermore, in order to increase therapeutic effect and to uniformly heat the malignant tumor tissue, magnetic magnetite is used for the magnetic fine particles including targeted magnetite particles prepared by coating the magnetic material with membrane having a particular affinity with surfaces of the malignant tumor cells. These particles can be targeted to specific molecular biomarkers of pathology using antibody-receptor interaction or by ionic charges.

The heating is due to magnetic hysteretic losses or frictional losses. Such heating may be employed for hyperthermia to kill cancer cells or to ablate tissues either by inducing coagulation necrosis or by increasing cell susceptibility to further treatments like chemotherapy or radiotherapy.

The heating or temperature rise in the tissue due to magnetic hysteresis losses is a complicated process that depends on numerous factors including the applied magnetic field, spatial variations within the tissue, the number and magnetic properties of the nanoparticles, tissue thermal properties, and blood perfusion. In order for hyperthermia to be effective, a certain thermal dosage equivalent cell exposure time must be achieved. Typically, low dose hyperthermia corresponds to a cumulative equivalent time at 43° C., t₄₃<50 minutes, and high or necrotic dosing corresponds to t₄₃>50-100 minutes. Unfortunately, there is no reliable, established means to a priori predict the temperature rise, the corresponding thermal dose, and the extent of tumor destruction and/or tissue damage, as many of the biophysical parameters are highly variable and change in response to hyperthermia (especially heat convection by blood flow). Temperature measurements using thermocouples are invasive and therefore should be avoided for in vivo situations. Magnetic resonance thermometry is expensive and there is also the potential for the AC electromagnetic fields inducing hyperthermia to affect the imaging procedure and much more significantly, the imaging magnetic field from MRI would profoundly affect the hyperthermia.

Another problem associated with magnetic hyperthermia is that targeting to a specific site might take several minutes or more depending on the targeting agent, local pathology, and blood flow conditions. It is difficult to predict if enough targeting has been achieved at a specific instant of time, and consequently whether sufficient magnetic particles have accumulated at the intended site to achieve the desired hyperthermia. Therefore, the appropriate time to initiate the magnetic field is not well known in advance. Furthermore, the magnetic field amplitude and treatment time need to be controlled to reach the appropriate thermal dose (heating/treatment of desired tissue) in the targeted area. Therefore, there is a need to monitor the hyperthermia heat pattern preferably using another non-invasive imaging modality. In addition, to facilitate the hyperthermia dosing, the magnetic power should to be regulated employing spatio-temporal measurements.

According to exemplary implementations of the present disclosure, disclosed herein is a method of magnetic hyperthermia control using ultrasound thermometry, the method comprising: acquiring a reference set of ultrasound data corresponding to a tissue of interest; applying a plurality of magnetic nanoparticles at the tissue of interest; applying an electromagnetic field based on a set of operating parameters to initiate the hyperthermia; acquiring another set of ultrasound data corresponding to the tissue of interest; and determining a temperature change based on the reference set of ultrasound data and the other set of ultrasound data.

Also disclosed herein in another embodiment is a system for magnetic hyperthermia control using ultrasound thermometry, the system comprising: a plurality of magnetic nanoparticles disposed substantially at a tissue of interest; an ultrasound system configured to provide at least thermometry data corresponding to the tissue of interest; a magnetic hyperthermia system configured to apply an electromagnetic field to the tissue of interest corresponding to a set of operating parameters; a controller in operable communication with the ultrasound system and the magnetic hyperthermia system, the controller configured to generate a command to the magnetic hyperthermia system to apply the electromagnetic field based on the at least thermometry data corresponding to the tissue of interest.

Further disclosed herein in another exemplary embodiment is a system for magnetic hyperthermia control using ultrasound thermometry, the system comprising: means for acquiring a reference set of ultrasound data corresponding to a tissue of interest; means for applying a plurality of magnetic nanoparticles at the tissue of interest; means for applying an electromagnetic field based on a set of operating parameters to initiate the hyperthermia; means for acquiring another set of ultrasound data corresponding to the tissue of interest; and means for determining a temperature change based on the reference set of ultrasound data and the other set of ultrasound data.

Also disclosed herein in yet another exemplary embodiment is a storage medium encoded with a machine readable computer program code, the code including instructions for causing a computer to implement the above mentioned method of magnetic hyperthermia control using ultrasound thermometry.

Further disclosed herein in another exemplary embodiment is a computer data signal, the computer data signal comprising instructions for causing a computer to implement the abovementioned method of magnetic hyperthermia control using ultrasound thermometry.

In another embodiment, there is disclosed herein a method of magnetic hyperthermia employing ultrasound imaging. The method includes: embedding a plurality of magnetic nanoparticles inside or on a plurality of microbubbles of a contrast agent; applying the plurality of magnetic nanoparticles at a tissue of interest; acquiring ultrasound data corresponding to the tissue of interest; and applying an electromagnetic field to initiate the hyperthermia.

In yet another embodiment of the invention there is disclosed herein a system for magnetic hyperthermia employing ultrasound imaging. The system includes: a plurality of magnetic nanoparticles embedded inside or on a plurality of microbubbles of a contrast agent; an ultrasound imaging system configured to provide imaging data corresponding to the tissue of interest; and a magnetic hyperthermia system configured to apply an electromagnetic field to the tissue of interest.

Additional features, functions and advantages associated with the disclosed methodology will be apparent from the detailed description which follows, particularly when reviewed in conjunction with the figures appended hereto.

To assist those of ordinary skill in the art in making and using the disclosed embodiments, reference is made to the appended figures, wherein like references are numbered alike:

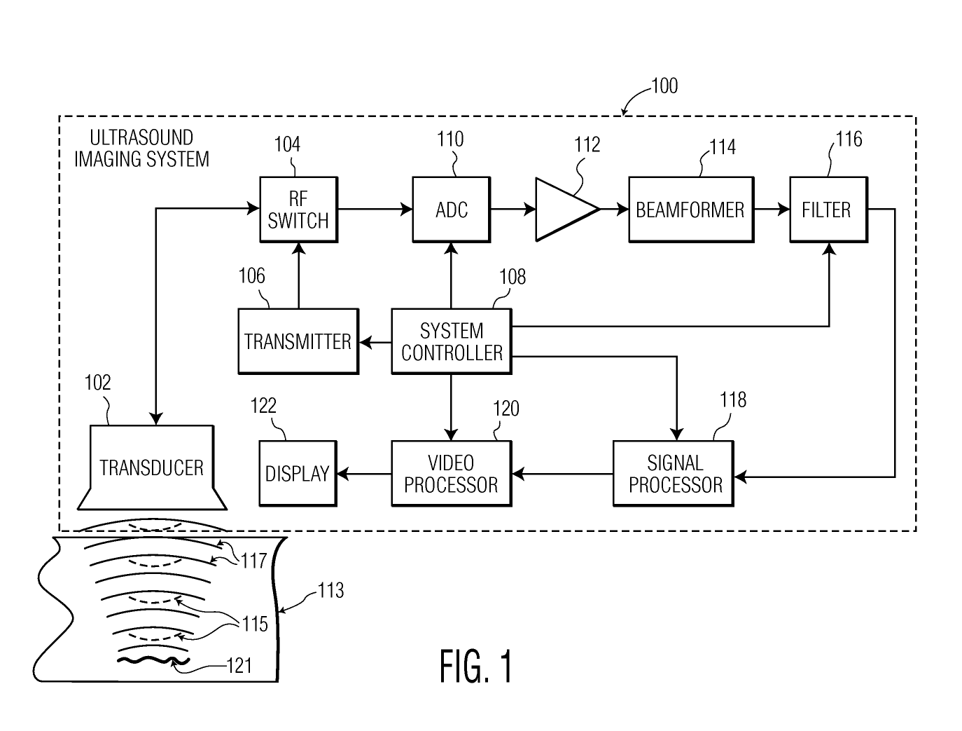

FIG. 1 depicts an ultrasound imaging system in accordance with an exemplary embodiment of the invention;

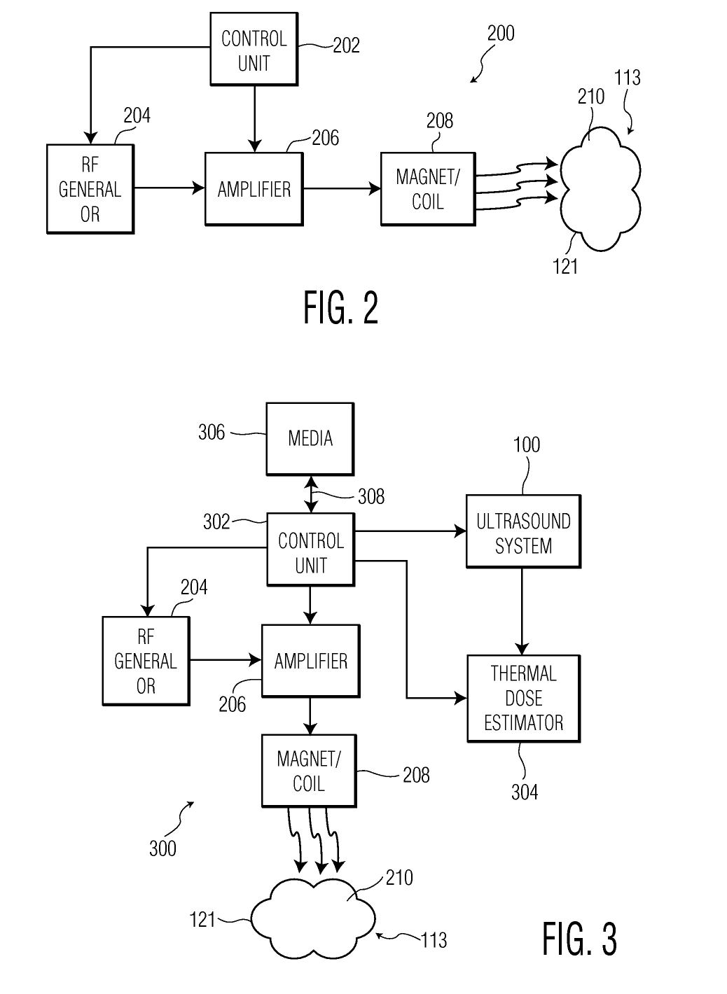

FIG. 2 a magnetic hyperthermia system as may be employed with an exemplary embodiment;

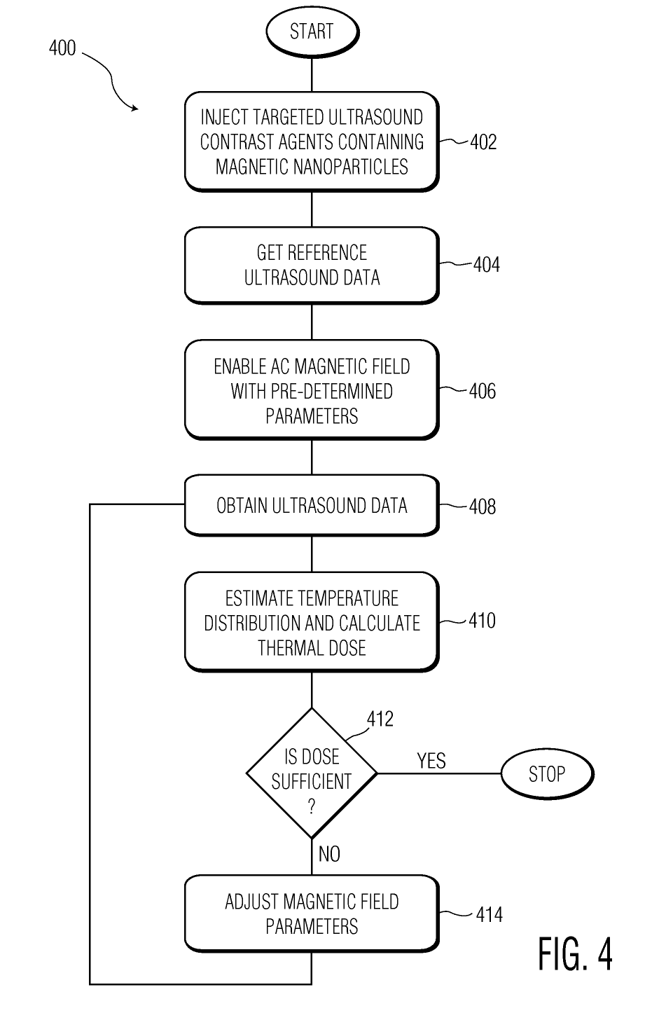

FIG. 3 is a block diagram depicting an integration of an ultrasound measuring and imaging system and a magnetic hyperthermia system in accordance with another exemplary embodiment of the invention;

FIG. 4 is a block diagram depicting a flowchart of a methodology for employing ultrasound thermometry to control magnetic hyperthermia in accordance with an exemplary embodiment.

As set forth herein in one or more exemplary embodiments, the present disclosure advantageously permits and facilitates a method and apparatus for ultrasound based monitoring of magnetic hyperthermia treatments, in particular, the temperature rise distribution and the associated cumulated thermal dose in affected tissues. It is well known that temperature affects ultrasonic properties of mammalian tissues, including, but not limited to, the speed of sound, attenuation, and the frequency-dependent wave scattering coefficient. In an exemplary embodiment, this temperature-dependence is utilized to create 2D/3D spatial maps of temperature elevations in the tissue. More particularly, 2D/3D spatial maps corresponding to the heating resultant from magnetic hyperthermia induced by the application of a magnetic field can be computed. Furthermore, the computed temperature profiles and received thermal dose are then employed to provide a feedback to the magnetic system to adjust the time of exposure, strength, frequency, and spatial position of the AC magnetic field, as well as changes in the dosage and composition of the injected bolus.

FIG. 1 depicts an ultrasound measuring and imaging system capable of viewing tissue and contrast agent(s) as may be adapted to and employed with an exemplary embodiment. In this regard, the ultrasound imaging system 100 may comprise a transducer 102, a RF switch 104, a transmitter 106, a system controller 108, an analog to digital converter (ADC) 110, a time gain control amplifier 112, a beamformer 114, a filter 116, a signal processor 118, a video processor 120, and a display 122. The transducer 102 may be electrically coupled to the RF switch 104. The RF switch 104 may be configured as shown with a transmit input coupled from the transmitter 106 and a transducer port electrically coupled to the transducer 102. The output of RF switch 104 may be electrically coupled to an ADC 110 before further processing by the time gain control amplifier 112. The time gain control amplifier 112 may be coupled to a beamformer 114. The beamformer 114 may be coupled to the filter 116. The filter 116 may be further coupled to a signal processor 118 before further processing in the video processor 120. The video processor 120 may then be configured to supply an input signal to a display 122. The system controller 108 may be coupled to the transmitter 106, the ADC 110, the filter 116, and both the signal processor 118 and the video processor 120 to provide necessary timing signals to each of the various devices.

As will be appreciated by persons having ordinary skill in the art, the system controller 108 and other processors, e.g., video processor 120, and signal processor 118 may include one or more processors, computers, and other hardware and software components for coordinating the overall operation of the ultrasonic imaging system 100. The RF switch 104 isolates the transmitter 106 of the ultrasound imaging system 100 from the ultrasonic response receiving and processing sections comprising the remaining elements illustrated in FIG. 1.

The system architecture illustrated in FIG. 1 provides an electronic transmit signal generated within the transmitter 106 that is converted to one or more ultrasonic pressure waves herein illustrated by ultrasound lines 115. When the ultrasound lines 115 encounter a tissue layer 113 that is receptive to ultrasound insonification the multiple transmit events or ultrasound lines 115 penetrate the tissue 113. As long as the magnitude of the multiple ultrasound lines 115 exceeds the attenuation affects of the tissue 113, the multiple ultrasound lines 115 will reach an internal target or tissue of interest 121, hereinafter referred to as tissue of interest. Those skilled in the art will appreciate that tissue boundaries or intersections between tissues with different ultrasonic impedances will develop ultrasonic responses at harmonics of the fundamental frequency of the multiple ultrasound lines 115.

As further illustrated in FIG. 1, such harmonic responses may be depicted by ultrasonic reflections 117. Those ultrasonic reflections 117 of a magnitude that exceed the attenuation effects from traversing tissue layer 113 may be monitored and converted into an electrical signal by the combination of the RF switch 104 and transducer 102. The electrical representation of the ultrasonic reflections 117 may be received at the ADC 110 where they are converted into a digital signal. The time gain control amplifier 112 coupled to the output of the ADC 110 may be configured to adjust amplification in relation to the total time a particular ultrasound line 115 needed to traverse the tissue layer 113. In this way, response signals from one or more tissues of interest 121 will be gain corrected so that ultrasonic reflections 117 generated from relatively shallow objects do not overwhelm in magnitude ultrasonic reflections 117 generated from insonified objects further removed from the transducer 102.

The output of the time gain control amplifier 112 may be beamformed, filtered and demodulated via beamformer 114, filter 116, and signal processor 118. The processed response signal may then be forwarded to the video processor 120. The video version of the response signal may then be forwarded to display 122 where the response signal image may be viewed. It will be further appreciated by those of ordinary skill in the art that the ultrasonic imaging system 100 may be configured to produce one or more images and or oscilloscopic traces along with other tabulated and or calculated information that would be useful to the operator.

FIG. 2 depicts a simplified magnetic hyperthermia system 200 as may be employed with an exemplary embodiment. The magnetic hyperthermia system 200 includes, but is not limited to a control unit 202 that controls the frequency of the RF signal generated by an RF generator 204 and the gain of an amplifier 206 needed to achieve a specific electromagnetic field strength produced by the magnet/coil 208.

In an embodiment the magnetic nanoparticles 210 may be embedded inside microbubbles used as ultrasound contrast agents (not shown), or attached outside thereto, in a manner so that it is possible to monitor by ultrasound the accumulation of the magnetic particles 210 to the particular site with a targeted agent. In another embodiment, the real time ultrasound data may then used to estimate temperature and thermal dose levels in the tissues of interest 121. The calculated thermal dose may then be utilized to provide feedback to a magnetic hyperthermia control system in order to adjust various operational parameters including, but not limited to the electromagnetic field strength, frequency, duration, and spatial distribution of the AC electromagnetic field. Optionally, in yet another embodiment, high intensity ultrasound may be used to destroy the microbubbles in the contrast agents and release embedded drugs after hyperthermia is achieved, to provide a combined magnetic hyperthermia and drug delivery capability and therapeutic effect.

As the magnetic nanoparticles 210 to be used in the present invention, any materials may be used so long as it absorbs electromagnetic energy to cause heat generating reaction and harmless to human body. It is particularly advantageous to use one that causes heat generating reaction by absorbing electromagnetic energy with frequencies that are difficultly absorbed by human body. Of these, ferromagnetic fine particles are preferably used since absorption efficiency of the electromagnetic wave is good, and for example, it may be exemplified by ceramics such as magnetite, ferrite, etc., or ferromagnetic metal such as permalloy, etc. Furthermore, the above-mentioned magnetic fine particles 210 are desirably having a particle size of about 5 micrometers or less, preferably about 1 micrometers or less.

FIG. 3 is a block diagram depicts an integration of an ultrasound measuring and imaging system 100 and a magnetic hyperthermia system 200 in accordance with another exemplary embodiment of the invention, now denoted by reference numeral 300. In one exemplary embodiment, a control unit 302 controls the frequency of the RF signal generated by an RF generator 204 to achieve a specific magnetic field strength produced by the magnet/coil 208. The control unit 302 also controls the ultrasound system 100 connected to and providing data to a thermal dosage estimator 304. The thermal dosage estimator 304 provides feedback to the control unit 302, which in turn, based on the thermal dosage estimator 304, controls the field strength and frequency of the AC magnetic field. It will be appreciated that while an exemplary embodiment is illustrated treating the thermal dosage estimator 304 as a separate process or function from the control unit 302, ultrasound system 100 and/or magnetic hyperthermial system 200, the functionality may be integrated anywhere. For example, in an exemplary embodiment, the controllers for each system 100, 200 and the thermal dosage estimator may be integrated into a single controller, process and function.

Continuing with FIGS. 1-3, in an exemplary embodiment, the transducer 102 of the ultrasound system 100 is preferably configured as an array to facilitate temperature determinations made during the magnetic hyperthermia process. In one exemplary embodiment the backscattered radio frequency (RF) signals are collected from the transducer 102. It has been shown that the time of flight of the ultrasonic waves change with tissue 113, 121 temperature. In fact, the changes in the speed of sound and the thermal dilation are linearly proportional to changes in temperature (denoted ΔT), with the proportionality constant being determined by the physical properties of the tissue 113,121. Therefore the measured time shifts in the RF signals between a reference situation and the heating phase may then be used monitor and control the magnetic hyperthermia as identified below.

In another embodiment the control unit 302 might be configured to adjust the spatial location of the magnetic coil based on the ultrasound feedback to better align with the tissue of interest 121. Since ultrasound imaging can provide real-time information on the location of the tissue of interest 121 requiring treatment, the AC magnet/coil 208 could be repositioned and moved to directly target the specific tissue of interest 121. As a result, in selected instances, it may be possible to reduce the volume of tissue 113 that is to be subjected to the AC electromagnetic field, and consequently the power requirements on the hyperthermia system 200 and the side effects to healthy tissue could be reduced or minimized.

In order to perform the prescribed functions and desired processing, as well as the computations therefore (e.g., the ultrasound control, magnetic hyperthermia control, and the like), the control unit 302, 202, the system controller 108, and other processors, e.g., video processor 120, signal processor 118, and the like may include, but not be limited to, a processor(s), computer(s), memory, storage, register(s), timing, interrupt(s), communication interface(s), and input/output signal interfaces, and the like, as well as combinations comprising at least one of the foregoing. In addition, the control unit 302, 202, the system controller 108, and other processors, e.g., video processor 120, signal processor 118, and the like may include signal interfaces to enable accurate sampling, conversion, acquisitions or generation of ultrasound signals as needed to facilitate thermal dose estimation and the like. Additional features of the control unit 302, 202, the system controller 108, and other processors, e.g., video processor 120, signal processor 118, and the like, are thoroughly discussed herein. It should also be noted that while a particular partitioning of functionality is depicted in the several figures for the purposes of describing the exemplary embodiments, such partitioning is illustrative only. The functionality of any of the various controllers, processors and the like may readily be partitioned and/or distributed in any fashion desired that facilitates practicing the disclosed embodiments.

In addition, it will be appreciated that the control unit, 202, 302 and/or system controller 108 and other processors may include software which comprises an ordered listing of executable instructions for implementing logical functions, which can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. The computer readable medium may be, for instance, an electronic, magnetic, optical, electromagnetic, infrared, or semiconductor system, apparatus, device, or propagation medium.

FIG. 4 depicts a flow chart illustrating the methodology of an exemplary embodiment shown generally as 400. In one embodiment the procedure starts as depicted at process block 402 with the administering of the bolus (e.g., either intra-venous or intra-arterial) containing a targeted contrast agents with embedded magnetic nanoparticles. Reference ultrasound data sets are obtained (possibly, and preferably in a continuous manner) as depicted at process block 404. The ultrasound data sets may include, but not be limited to, a raw RF dataset or signal processed A-line data, or B-mode images. To start the hyperthermia process as depicted at process block 406, an AC magnetic field is applied employing a selected initial set of parameters for the magnetic field strength and frequency and the like. While an initial set of parameters is desired to initiate the hyperthermia close to the desired dosage, it is not necessary. In fact, advantageously the disclosed embodiments particularly address the ambiguity of selection of the initial field strength and frequency. By starting at essentially arbitrarily low setting for the parameters, the closed loop nature of the system disclosed will automatically compensate. When the thermometry data indicates that more/less dosage is needed, the electromagnetic field is automatically adjusted. This approach essentially eliminates the ambiguity inherent in existing hyperthermia applications for selecting the initial field strength and duration.

Substantially concurrent with the magnetic field application of process block 406, ultrasound data are obtained as depicted at process block 408. The ultrasound data are used with the reference data state(s) to facilitate determining a temperature distribution, and thereby the thermal dose within the tissue as depicted at process block 410. The dosage levels are then calculated for various points in the tissue. If the dosing is not sufficient, the magnetic field parameters are adjusted and process continues until the required dosage levels are achieved as depicted at decision block 412 and process block 414. Of course, if the dosage is sufficient, the magnetic field is removed and the process terminated.

Optionally, in yet another exemplary embodiment, the magnetic nanoparticles can be embedded in a microbubble used as ultrasound contrast agent. A higher intensity ultrasound field can be applied to destroy the microbubbles that may release a therapeutic drug.

It will further be appreciated that while particular sensors and nomenclature are enumerated to describe an exemplary embodiment, such sensors are described for illustration only and are not limiting. Numerous variations, substitutes, and equivalents will be apparent to those contemplating the disclosure herein.

In sum, the disclosed invention advantageously permits and facilitates controlled magnetic hyperthermia including in some embodiments ultrasonic temperature sensing and feedback. Furthermore, the disclosed system and methodology may be particularly directed to cancer treatment or ultrasound monitored therapeutic applications. For example, embodiments of the invention may include targeted therapeutic drug applications at a specified site, particularly where the sensitivity of the targeted tissues/tumor has been increased due to hyperthermia applications. The disclosed system and methodologies provide significant benefits to operators, particularly physicians, relying on effectively uncontrolled “iterative” processes for magnetic hyperthermia dosing. Indeed, the disclosed system and methodology provides measurement and control means particularly addressing feedback control of the hyperthermia processes. An additional advantage of the disclosed system and methodologies is that the magnetic hyperthermia can be performed based on more accurate hyperthermia dosing resulting in lowered patient dosages with reduced impact to adjacent untargeted tissues.

The system and methodology described in the numerous embodiments hereinbefore provides a system and methods to for apparatus for ultrasound based monitoring of magnetic hyperthermia treatments, in particular, the temperature rise distribution in affected tissues. In addition, the disclosed invention may be embodied in the form of computer-implemented processes and apparatuses for practicing those processes. The present invention can also be embodied in the form of computer program code containing instructions embodied in tangible media 306, such as floppy diskettes, CD-ROMs, hard drives, or any other computer-readable storage medium, wherein, when the computer program code is loaded into and executed by a computer, the computer becomes an apparatus for practicing the invention. The present invention can also be embodied in the form of computer program code, for example, whether stored in a storage medium, loaded into and/or executed by a computer, or as data signal 308 transmitted whether a modulated carrier wave or not, over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

It will be appreciated that the use of “first” and “second” or other similar nomenclature for denoting similar items is not intended to specify or imply any particular order unless otherwise specifically stated. Likewise the use of “a” or “an” or other similar nomenclature is intended to mean “one or more” unless otherwise specifically stated.

While the invention has been described with reference to a exemplary embodiments thereof, it will be understood by those skilled in the art that the present disclosure is not limited to such exemplary embodiments and that various changes may be made and equivalents may be substituted for elements thereof without departing from the scope of the invention. In addition, a variety of modifications enhancements and/or variations may be made to adapt a particular situation or material to the teachings of the invention without departing from the essential spirit or scope thereof. Therefore, it is intended that the invention not be limited to the particular embodiment disclosed as the best mode contemplated for carrying out this invention, but that the invention will include all embodiments falling within the scope of the appended claims.

1. A method of magnetic hyperthermia control using ultrasound thermometry, the method comprising:
acquiring a reference set of ultrasound data corresponding to a tissue of interest (121);
applying a plurality of magnetic nanoparticles (210) at said tissue of interest (121);
applying an electromagnetic field based on a set of operating parameters to initiate the hyperthermia;
acquiring another set of ultrasound data corresponding to said tissue of interest (121); and
determining a temperature change based on said reference set of ultrasound data and said another set of ultrasound data.

2. The method of claim 1 further including:
adjusting said set of operating parameters and applying another electromagnetic field based on said adjusted set of operating parameters, if, based on said temperature change, a dosage corresponding to said applying an electromagnetic field is insufficient; otherwise
terminating said applying.

3. The method of claim 1 wherein said applying an electromagnetic field is at least one of initiated by a user or automatically initiated based on feedback from said determining.

4. The method of claim 1 further including: embedding said magnetic nanoparticles (210) inside or on a plurality of microbubbles of an ultrasound contrast agent.

5. The method of claim 1 further including: applying high intensity ultrasound energy to destroy microbubbles of an ultrasound contrast agent.

6. The method of claim 5 further including: delivering a medication or drug via said microbubbles of said ultrasound contrast agent.

7. The method of claim 1 further including: repositioning a means of generating said electromagnetic field (208) to reduce a volume of tissue (113) that is subjected to said applying said electromagnetic field.

8. The method of claim 7 wherein said repositioning and reducing said volume results in a reduction of power otherwise required for said applying said electromagnetic field.

9. The method of claim 7 wherein said repositioning facilitates an enhanced alignment with said tissue of interest (121).

10. A system (300) for magnetic hyperthermia control using ultrasound thermometry, the system (300) comprising:
a plurality of magnetic nanoparticles (210) disposed substantially at a tissue of interest (121);
an ultrasound system (100) configured to provide at least thermometry data corresponding to said tissue of interest (121);
a magnetic hyperthermia system (200) configured to apply an electromagnetic field to said tissue of interest corresponding to a set of operating parameters;
a controller (302) in operable communication with said ultrasound system (100) and said magnetic hyperthermia system (200), said controller (302) configured to generate a command to said magnetic hyperthermia system (200) to apply said electromagnetic field based on said at least thermometry data corresponding to said tissue of interest (121).

11. The system (300) of claim 10 further including:
said controller (302) adjusting said set of operating parameters and generating another command to said magnetic hyperthermia system (200) applying another electromagnetic field based on said adjusted set of operating parameters if, based on said temperature change, a dosage corresponding to said applying an electromagnetic field is insufficient; otherwise
terminating said applying.

12. The system (300) of claim 10 wherein said application of said electromagnetic field is at least one of initiated by a user or automatically initiated based on feedback from said ultrasound system (100).

13. The system (300) of claim 10 wherein said magnetic nanoparticles (210) are embedded inside or on a plurality of microbubbles of an ultrasound contrast agent.

14. The system (300) of claim 10 further including, said controller (302) generating a command to said ultrasound system to apply high intensity ultrasound energy to destroy microbubbles of an ultrasound contrast agent.

15. The system (300) of claim 14 wherein said microbubbles of said ultrasound contrast agent are configured to deliver a medication or drug.

16. The system (300) of claim 10 wherein a means of generating said electromagnetic field (208) of said magnetic hyperthermia system (200) is repositioned to reduce a volume of tissue (113) that is subjected to said electromagnetic field.

17. The system (300) of claim 16 wherein said repositioning and reducing said volume results in a reduction of power otherwise required for said electromagnetic field.

18. The system (300) of claim 16 wherein said repositioning facilitates an enhanced alignment with said tissue of interest (121).

19. A system (300) for magnetic hyperthermia control using ultrasound thermometry, the system (300) comprising:
means for acquiring a reference set of ultrasound data corresponding to a tissue of interest (121);
means for applying a plurality of magnetic nanoparticles (210) at said tissue of interest (121);
means for applying an electromagnetic field based on a set of operating parameters to initiate the hyperthermia;
means for acquiring another set of ultrasound data corresponding to said tissue of interest (121); and
means for determining a temperature change based on said reference set of ultrasound data and said another set of ultrasound data.

20. A storage medium (306) encoded with a machine readable computer program code, the code including instructions for causing a computer to implement a method of magnetic hyperthermia control using ultrasound thermometry, the method comprising:
acquiring a reference set of ultrasound data corresponding to a tissue of interest (121);
applying a plurality of magnetic nanoparticles (210) at said tissue of interest (121);
applying an electromagnetic field based on a set of operating parameters to initiate the hyperthermia;
acquiring another set of ultrasound data corresponding to said tissue of interest (121); and
determining a temperature change based on said reference set of ultrasound data and said another set of ultrasound data.

21. A computer data signal, the computer data signal (308) comprising instructions for causing a computer to implement a method of magnetic hyperthermia control using ultrasound thermometry, the method comprising:
acquiring a reference set of ultrasound data corresponding to a tissue of interest (121);
applying a plurality of magnetic nanoparticles (210) at said tissue of interest (121);
applying an electromagnetic field based on a set of operating parameters to initiate the hyperthermia;
acquiring another set of ultrasound data corresponding to said tissue of interest (121); and
determining a temperature change based on said reference set of ultrasound data and said another set of ultrasound data.

22. A method of magnetic hyperthermia employing ultrasound imaging comprising:
embedding a plurality of magnetic nanoparticles (210) inside or on a plurality of microbubbles of a contrast agent;
applying said plurality of magnetic nanoparticles (210) at a tissue of interest (121);
acquiring ultrasound data corresponding to said tissue of interest (121); and
applying an electromagnetic field to initiate the hyperthermia.

23. The method of claim 22 wherein said ultrasound data is employed to generate images indicative of a concentration of said plurality of magnetic nanoparticles (210) at said structure of interest (121).

24. The method of claim 22 further including, delivering a medication or drug via said plurality of microbubbles of said contrast agent by applying high intensity ultrasound energy to destroy said plurality of microbubbles.

25. A system for magnetic hyperthermia employing ultrasound imaging, the system comprising:
a plurality of magnetic nanoparticles (210) embedded inside or on a plurality of microbubbules of a contrast agent;
an ultrasound imaging system (100) configured to provide imaging data corresponding to said tissue of interest (121); and
a magnetic hyperthermia system (200) configured to apply an electromagnetic field to said tissue of interest (121).

26. The system of claim 25 wherein said imaging data is employed to ascertain a presence of said plurality of magnetic nanoparticles (210) disposed substantially at said tissue of interest (121).

27. The system of claim 25 further including: a medication or drug delivered via said microbubbles of said contrast agent by applying high intensity ultrasound energy to destroy the microbubbles.
